(19) United States
(12) Patent Application Publication
(10) Pub. No.: US 2008/0058839 A1
(43) Pub. Date: Mar. 6, 2008

(54) Reverse tapered guidewire and method of use

(76) Inventors: Anthony A. Nobles, Fountain Valley, CA (US); Alan W. Heldman, Miami Beach, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/702,915

(22) Filed: Feb. 6, 2007

Related U.S. Application Data
(60) Provisional application No. 60/771,522, filed on Feb. 7, 2006.

(51) Int. Cl. A61B 17/04

(52) U.S. Cl. 606/148; 128/898

(57) ABSTRACT

A reverse tapered guidewire may comprise a proximal segment and a distal segment. The proximal segment may have a cross-sectional diameter smaller than a cross-sectional diameter of the distal segment. The guidewire may be inserted through an access site on a patient. A medical device may be advanced over the proximal segment of the guidewire. After the medical device is withdrawn from the patient, the guidewire may be used to re-access the site.

### CROSS-REFERENCE TO RELATED APPLICATIONS

This application claims the benefit of U.S. provisional application No. 60/771,522, filed Feb. 7, 2006, the entirety of which is hereby incorporated by reference.

### BACKGROUND OF THE INVENTION

1. Field of the Invention

Certain embodiments of the present invention relate to a medical guidewire for advancing intraluminal medical devices, such as a suturing device, within a body lumen. More particularly, preferred embodiments relate to a reverse tapered guidewire and method of use for advancing the guidewire through a body lumen and providing re-access to an endoluminal cavity throughout the medical procedure.

2. Description of the Related Art

Physicians frequently use sutures to close cuts, punctures, incisions and other openings in various biological tissue, such as blood vessels, of the human body.

In an arterial catheterization procedure, a relatively small percutaneous incision is made in the femoral or other artery. A catheter is inserted through the incision and directed along an arterial path to a target area, such as the heart, to perform one or more procedures, such as an angioplasty or angiogram. These procedures are intended to be relatively quick ‘outpatient’ procedures.

Upon completion of the catheterization procedure, the physician typically creates a ‘thrombus patch’ by applying direct pressure to the patient's thigh to make the blood around the incision clot. It is very important that the applied pressure does not impede the flow of blood through the femoral artery. As a result, it is commonplace for the physician to apply direct pressure by hand for the first twenty minutes after the procedure. During this time, the physician can feel the pulse to assure the artery is not occluded. Afterwards, the physician typically transfers responsibility to an assistant who then applies direct pressure using sandbags, clamps or other devices. A significant problem with this approach is that it is frequently necessary to apply the pressure for an extended period of time, such as twenty-four hours or longer.

Another problem with the thrombus patch method is that the high blood pressure in the artery can cause the thrombus patch to rupture or burst while direct pressure is being applied to the thigh or after direct pressure is removed. This requires the entire process to be reinitiated. If the patch ruptures and is not quickly restored, substantial bleeding can occur, with potentially fatal consequences. Because thrombus patches frequently burst, the patient is often kept in the hospital or catheterization lab overnight for observation. As a result, these ‘out-patient’ procedures become ‘in-patient’ procedures, simply because a thrombus patch is often unreliable and/or difficult to create. Staying in the hospital increases patient discomfort and hospital expenses, which are often disproportionate to the actual medical procedure performed.

Furthermore, if a thrombus patch cannot be adequately formed, the physician may need to anesthetize the patient and occlude the blood flow to the artery. At this point, the physician is required to make a large incision in the thigh to allow conventional suturing with a needle, suture the artery with conventional means, restore blood flow to the artery, and suture the incision in the thigh. This results in additional discomfort and expenses for the patient.

While the above problems could potentially be avoided by suturing the blood vessel immediately following the catheterization procedure, the size and location of the artery make suturing extremely difficult. More specifically, the opening in the thigh is often too small and too deep to provide enough working space for suturing the artery using conventional methods. Thus, in order to suture the vessel using conventional methods, the opening in the thigh would have to be significantly enlarged, thereby further increasing the recovery period and exposing the patient to additional discomfort, undesirable scarring, possible infection and other health risks.

### SUMMARY OF THE INVENTION

Certain embodiments of the invention are directed to a reverse tapered guidewire and method of use. Preferred embodiments relate to the use of the guidewire to suture an opening in a body of a patient, and to re-access the opening.

In U.S. Pat. No. 6,117,144, U.S. Pat. No. 6,562,052 and U.S. patent application Ser. No. 11/235,751, filed Sep. 27, 2005 and published on Mar. 30, 2006 as U.S. Patent Publication No. 2006/0069397, each incorporated herein in their entireties by reference, methods and devices are disclosed for closing incisions, or other openings, within biological tissue, for example by suturing biological tissue, such as an organ or blood vessel. These methods are particularly well suited for suturing an incision or puncture made in an artery, such as the femoral artery, following a catheterization procedure. This method of suturing the blood vessel immediately following the catheterization procedure eliminates the need to apply pressure to a patient's thigh for an extended period of time, and eliminates many of the complications and costs associated with the creation of a thrombus patch. One suitable suturing device is the SuperStitch® closure device available from Sutura, Inc. of Fountain Valley, Calif.

In one embodiment of a catheterization procedure, a guidewire is first advanced through the puncture in the wall of a blood vessel. An introducer may be advanced over the guidewire into the blood vessel, and the guidewire may then be removed, or alternatively, may remain in the blood vessel and used to deliver therapy or other devices. After the desired treatment has been completed, if the guidewire has been removed, the suturing device may be delivered through the introducer to deploy the sutures to close the puncture. Alternatively, if the guidewire has remained or is reintroduced into the vessel through the introducer, the suturing device may be delivered over the guidewire into the blood vessel to deploy the sutures.

In one embodiment of using a suturing device such as the SuperStitch® closure device, for the device to properly place the sutures across the puncture site, after the suturing device has been delivered into the blood vessel, the introducer is pulled proximally just out of the blood vessel so as not to interfere with deployment of the device.

However, the sutures may not always be successful in sealing the puncture in the blood vessel. For example, in patients with extensive plaque build up along the arterial wall, the sharp edges of the plaque may cut the suture as it is being pulled tight about the opening. Alternatively, in certain patients with thin arterial walls, the force of the suture against the arterial wall as it is being pulled tight may cause the suture to be pulled through the wall and thus prevent closure of the wall opening. In such instances, it would be advantageous to be able to re-access the incision with a suture delivery device to apply another suture to the incision. But with the introducer removed from the vessel, redelivery can be difficult.

In addition, during certain procedures, pre-loading of the sutures may be desired. For example, a larger incision may be needed to perform the medical procedure. In such instances, it would be advantageous to be able to pre-load the sutures at the incision site, without tying or knotting the sutures, and maintain access to the incision after the sutures have been placed to re-access the incision and perform the medical procedure. Again, with introducer removed from the vessel, re-access may be difficult.

In other procedures, it may be desirable to reinsert a suturing device with a different orientation (e.g., rotated 90 degrees) for the placement of additional sutures.

In keeping with the foregoing discussion, one embodiment of the present invention provides an improved guidewire for use with intraluminal medical devices, particularly for use with a suturing device for remotely sealing an incision in a blood vessel or other body tissue. The guidewire has a tapered proximal end or reverse taper, wherein a proximal portion of the guidewire is smaller than a distal portion of the guidewire, for allowing catheter and/or medical devices to be advanced and withdrawn over the wire while the guidewire is left in place inside the incision in the blood vessel. This includes the ability to advance medical devices, for example a suture delivery device or other intraluminal medical device, having small diameter lumens over the proximal end of the guidewire and sufficiently far into the patient's body to access the incision site or other desired locations. The guidewire may also desirably remain in place, providing continuous access to the blood vessel incision during and after delivery of the suturing device. For example, an introducer that has been intentionally or unintentionally removed from a blood vessel may be reinserted into the blood vessel over the guidewire using an obturator. Thus, the suturing device may be delivered to the intraluminal treatment site, removed and subsequently re-introduced to the treatment site over the guidewire one or more times as necessary to completely close the incision.

The tapered proximal end of the guidewire may comprise a gradual taper, wherein the guidewire gradually decreases in dimension from a distal location toward a proximal location. The taper may also comprise a stepped down portion, wherein a proximal portion of the guidewire has a first diameter and a distal portion of the guidewire has a second, larger diameter.

The guidewire, even with the proximal taper, desirably has sufficient “pushability” to maneuver through the patient's blood vessels or bodily tissue to the desired treatment site. Accordingly, a distal region of the guidewire may have a larger cross-sectional diameter than a proximal region in order to provide sufficient rigidity for pushability and trackability and may at the same time be flexible enough to prevent trauma to the blood vessel walls. However, a proximal region may have a smaller diameter, yet remain sufficiently stiff so that it will not buckle when advanced and will provide sufficient trackability to devices provided thereover.

In one embodiment, a method of delivering an intravascular medical device to a body lumen of a patient is provided. A guidewire may be advanced through an access site on the patient into the lumen of a blood vessel. The guidewire may have a proximal region and a distal region. The proximal region may have a cross-sectional diameter smaller than a cross sectional diameter of the distal region. An intravascular device may be advanced over at least the proximal region of the guidewire into the lumen of the blood vessel. An operation may be performed at a treatment site within the patient with the intravascular device. The intravascular device may be withdrawn from the patient. The same or a different intravascular device may be introduced over the proximal segment of the guidewire.

In another embodiment, a method of providing access to a blood vessel of a patient is provided. A guidewire may be advanced through an access site in the patient's tissue and through an incision in a lumen of a blood vessel. The guidewire may have a proximal region and a distal region. The proximal region may have a cross-sectional diameter smaller than a cross sectional diameter of the distal region. A proximal portion of the wire may be held outside of the patient to maintain the position of the distal region of the guidewire within the incision in the blood vessel. A medical device may be advanced over the proximal region of the guidewire until it abuts the beginning of the larger diameter distal region. The guidewire and the medical device may be moved distally through the patient's tissue past the incision in the body lumen.

In another embodiment, a method of performing a medical procedure is provided. A guidewire may be delivered into a patient, the guidewire having a proximal segment and a distal segment, wherein the proximal segment has a smaller diameter than that of the distal segment, such that the proximal segment extends outside the patient after the guidewire is delivered. A medical device may be advanced over the guidewire into the patient.

In another embodiment, a method of placing at least one suture is provided. A guidewire may be inserted into a body opening. The guidewire may comprise a proximal segment and a distal segment, the proximal segment having a smaller diameter than the distal segment. A suture delivery device may be advanced at least partially over the proximal segment of the guidewire to the opening. At least one suture may be applied to the opening using the suture delivery device. The suture delivery device may be withdrawn from the patient's body over the guidewire while retaining the guidewire within the body opening.

In one embodiment, a guidewire may comprise a proximal segment and a distal segment, wherein the proximal segment has a smaller diameter than that of the distal segment. The proximal segment may have a substantially constant diameter and substantially consistent mechanical properties from a proximal end of the guidewire to a transition between the proximal and distal segments.

In one preferred embodiment, a method of advancing a medical device over a guidewire is provided. Typically, a guidewire is inserted into the patient's blood vessel via an access site and advanced to a treatment site. One or more catheters or other medical devices may then be advanced over the guidewire to the treatment site to provide a desired treatment. The guidewire is preferably left in place during the delivery of the medical device(s) and the treatment. The guidewire may have a constant diameter, or may have a proximal region with a smaller diameter cross-section and allows the medical device(s) to be advanced to the treatment site over the guidewire via lumen(s) in the medical device(s). Once the treatment has been performed, the medical device(s) may be withdrawn over the guidewire. Here, the guidewire is still in position, providing access to the treatment site. Alternatively, the guidewire may be removed and may be replaced with another guidewire. Thus, if for example the first treatment fails, or if additional treatments are needed, the medical device, or another medical device, may be readvanced over the guidewire to re-access the original treatment site and continue treatment.

In another embodiment, a method of advancing a suture delivery device over a guidewire is provided. The method comprises inserting a reverse tapered guidewire into an incision in a blood vessel or other body lumen. A suture delivery device comprising an elongate body having a lumen, at least one needle and a suture catch assembly which extends from the distal portion of the body and releasably holds at least one suture is advanced over a narrow proximal region of the guidewire into the blood vessel opening. The needles are then deployed from and then retracted into the body, during which time the needles pierce the vessel wall on opposite sides of the incision, release and capture suture ends from arms of the suture catch assembly, and pull the ends of the suture proximally through the vessel wall. The arms are then moved to a retracted position, and the device is withdrawn from the blood vessel and from the patient's body over the guidewire. The same or a different suture delivery device may then be re-loaded and re-inserted over the guidewire and access the same incision in the blood vessel wall, for example to delivery multiple sutures to a large incision or to deliver another suture to incision should the first suture fail.

In another embodiment, a method of pre-loading at least one suture at an incision site is provided. The method comprises inserting a reverse taper guidewire into an incision in a patient's blood vessel or other body lumen. A suture delivery device may then be advanced over the narrow proximal portion of the guidewire to the incision site. The narrow proximal portion of the guidewire may be operatively sized to allow the suture delivery device to be advanced to the incision site. The suture delivery device may then deliver the sutures and then be withdrawn from the blood vessel and the patient's body over the guidewire. The sutures may then be laid aside without being pulled closed and tied and the guidewire may be used to re-access the incision site to perform a medical procedure within the blood vessel. In certain embodiments, the doctor may introduce a larger catheter over the guidewire to enlarge the incision in the blood vessel in order to perform the medical procedure. Once the medical procedure is complete, the pre-loaded sutures may be pulled tight and knotted to close the enlarged incision.

All of these embodiments are intended to be within the scope of the present invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 illustrates a guidewire according to one embodiment of the present invention.

FIG. 2 is a schematic view of one embodiment of a guidewire.

FIG. 3 is a schematic view of another embodiment of a guidewire

FIG. 4 is a schematic view of another embodiment of a guidewire

FIG. 5 illustrates one embodiment of a suture delivery device.

FIG. 6A illustrates a suture introducer head having suture arms and needles retracted into the device housing.

FIGS. 6B and 6C illustrate a suture introducer head having suture arms partially extended.

FIG. 7 illustrates an exemplifying use environment, such as a patient's thigh.

FIG. 8 illustrates a guidewire extending into a lumen, such as a blood vessel, with an obturator and an introducer extending over into the lumen.

FIG. 9 illustrates an embodiment of a reverse tapered guidewire extending through an introducer into a lumen, such as a blood vessel, with a transition between a proximal segment and a distal segment being located outside the introducer and the lumen. A medical device, such as a suturing device, is shown positioned over a proximal segment of the guidewire.

FIG. 10 illustrates the guidewire of FIG. 9 with the medical device positioned over the proximal segment of the guidewire and the medical device extending through the introducer into the lumen. The introducer is shown withdrawn from the lumen.

FIG. 11 illustrates the guidewire of FIGS. 9-10 and a suture disposed through a tissue portion and extending through the introducer.

FIG. 12 illustrates the guidewire of FIGS. 9-11 and an obturator about to be inserted into the introducer over the guidewire.

FIG. 13 illustrates the guidewire of FIGS. 9-12 with the obturator and the introducer extending at least partially over a distal segment of the guidewire into the lumen.

FIG. 14 illustrates the guidewire of FIGS. 9-13 with a medical device, such as a suturing device, positioned over the proximal segment of the guidewire.

FIG. 15 illustrates the guidewire of FIGS. 9-14 with the medical device positioned over the proximal segment of the guidewire and the medical device extending through the introducer into the lumen. The introducer is shown withdrawn from the lumen.

FIG. 16 is a cross-sectional view of a distal end of a suturing device disposed in a blood vessel.

FIG. 17 is a cross-sectional view of the suturing device of FIG. 16 with suture clasp arms partially extended.

FIG. 18 is a cross-sectional view of the suturing device of FIGS. 16 and 17 with suture clasp arms fully extended and needles deployed for engaging suture ends.

FIG. 19 illustrates an embodiment of a reverse tapered guidewire extending through an introducer into a lumen, such as a blood vessel; a suture disposed through a tissue portion and extending through the introducer; and a knot placement device having a threader loaded therein. A pair of suture ends is shown extending through the threader.

FIG. 20 illustrates the knot placement device positioned through the introducer over the suture.

### DETAILED DESCRIPTION OF THE PREFERRED EMBODIMENTS

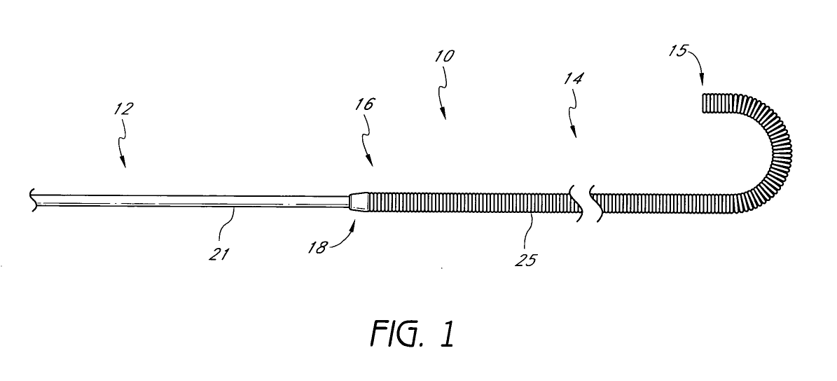

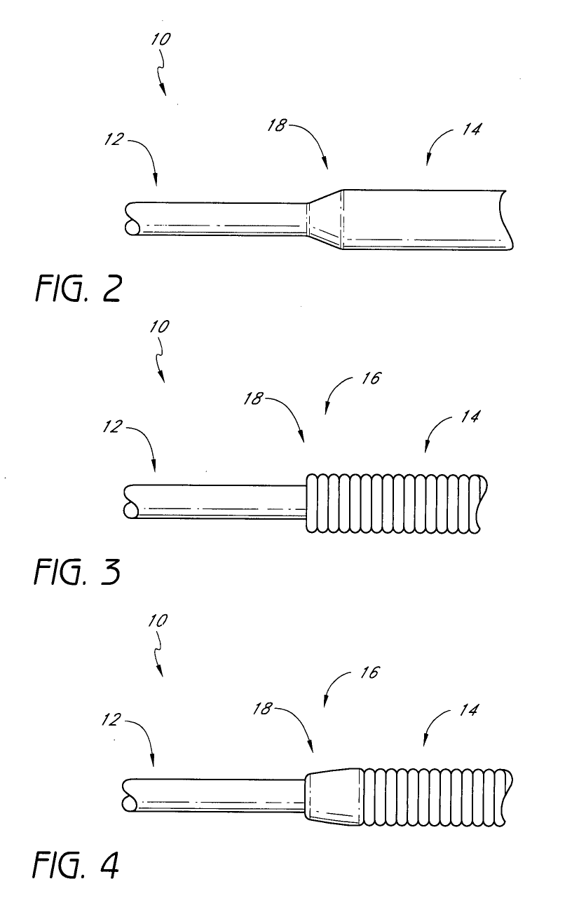

FIG. 1 illustrates an embodiment of a reverse tapered guidewire 10, having a distal segment 14 and a proximal segment 12. As used herein, the term “reverse tapered” refers to the guidewire being smaller in the proximal segment 12 than in the distal segment 14 The guidewire 10 need not necessarily have a gradual transition 18 from the distal segment 14 to the proximal segment 12. Thus, in one embodiment, illustrated in FIG. 3, the guidewire 10 may have a distinct step between the proximal segment 12 and distal segment 14, but have a constant or substantially constant cross-section between the proximal end and the transition 18 between the proximal and distal segments. In other embodiments, a gradual taper may be provided, as shown in FIGS. 2 and 4.

In one embodiment, the length of the guidewire 10 ranges from about 50 cm to about 320 cm, more typically ranging from about 120 cm to about 200 cm, and preferably from about 175 cm to about 190 cm for the coronary anatomy or alternatively from about 120-170 cm for accessing a femoral artery. In other embodiments, the length of the guidewire 10 may range from about 40 cm or less to about 120 cm or more, including about 70 cm or less to about 100 cm or more. In one preferred embodiment, the guidewire 10 may be about 82.6 cm long. The length and diameter of guidewire 10 may be varied to suit the particular procedures in which it is to be used and the materials from which it is constructed.

The guidewire is preferably made of superelastic nickel-titanium or nitinol, or may be made of stainless steel or other suitable material.

The distal segment 14 of the guidewire is configured to be advanced through a blood vessel or lumen within the patient and has a cross-sectional diameter sufficient to provide the stiffness and pushability necessary to advance the guidewire through the blood vessel or body lumen. For example, the distal segment of the guidewire may have a cross-sectional diameter ranging between about 0.030 inches or less to about 0.050 inches or more, more preferably between about 0.035 inches or less to about 0.040 inches or more, and even more preferably about 0.038 inches, such that the guidewire may be compatible with medical devices having a 8 Fr lumen or larger.

In certain embodiments, the distal segment 14 of the guidewire may be comprised of an elongate core surrounded by a helical coil, as described further below, in order to provide the rigidity necessary to push the guidewire through a blood vessel while ensuring that the guidewire remain flexible enough to prevent trauma or damage to the blood vessel walls as it is being advanced through the blood vessel. In alternative embodiments, the distal segment of the guidewire may be constructed using techniques known to those skilled in the art and may further include an elongate core, a helical coil covering the elongate core, a tapered distal end, a flexible tip and/or other suitable features for facilitating advancing and positioning the guidewire within the body lumen.

In some embodiments, the distal segment 14 of the guidewire may be constructed using techniques known to those skilled in the art to provide sufficient trackability to permit an introducer and an obturator to be inserted over the guidewire into a lumen.

The distal segment 14 of the guide wire may be of a suitable length for advancing the guidewire from the access site, for example the femoral artery, through the blood vessel or body lumen to the site of a medical procedure. In some embodiments, the length of the distal segment may range from about 10 cm or less to about 200 cm or more, including about 30 cm, about 60 cm, about 90 cm, about 120 cm, and about 150 cm. In one embodiment, the distal segment of the guidewire is about 50 cm long.

The proximal segment 12 of the guidewire has a cross-sectional diameter smaller than the cross-sectional diameter of the distal segment, for example the cross-sectional diameter of the proximal segment may range from between about 0.010 inches or less to about 0.018 inches or more, more preferably between about 0.012 inches or less to about 0.014 inches or more, even more preferably about 0.010 inches such that the proximal segment may be compatible with medical devices having a 7 Fr lumen or smaller, alternatively a 6 Fr lumen or smaller, alternatively a 5 Fr lumen or smaller. Advantageously, the smaller proximal segment of the guidewire allows use of devices with smaller lumens, particularly those that can be advanced over the smaller proximal segment 12 but not the larger distal segment 14.

The proximal segment 12 has a length typically ranging about 1 cm or less to about 30 cm or more, more preferably from about 2 cm or less to about 20 cm or more, even more preferably from about 2 cm or less to about 10 cm or more, although longer segments may be used as needed to provide sufficient length to advance a medical device over the narrow proximal segment 12 to the incision, the treatment site or other desired locations. In some embodiments, the length of the proximal segment 12 may range from about 10 cm or less to about 100 cm or more, including about 20 cm, about 40 cm, about 60 cm, and about 80 cm. In one embodiment, the proximal segment 12 may be about 32 cm long.

When used with a suturing device, the length of the proximal segment is sufficient to extend at least from outside of the patient to a location distal to the incision within the blood vessel.

This smaller diameter of the proximal segment 12 allows a medical device having a small lumen, for example a 6 Fr, 5 Fr, 4 Fr or smaller lumen, to be loaded on the narrow proximal segment of a guidewire having a distal segment positioned at least partially in the body lumen. Here, the medical device may be advanced over both the proximal and distal segments of the guidewire to the treatment site. Alternatively, the medical device can only be advanced over the proximal segment to be located at a different desired treatment location, such as an incision site. In either case, the guidewire may remain in place during the medical procedure and provide re-access to the incision after the medical device is removed should it be necessary.

In one embodiment, as shown in FIG. 1, a tapered guidewire 10 may comprise a stiff elongate core 21 extending the entire length or substantially the entire length of the guidewire and having a cross-sectional diameter ranging from between about 0.010 inches or less to about 0.018 inches or more, more preferably between about 0.012 inches or less to about 0.014 inches or more, and even more preferably about 0.010 inches. The distal segment 14 of the elongate core member 21 may be wrapped with a helical coil 25 or a tubular body of polymeric material according to methods known to those skilled in the art to provide a distal segment 14 having a cross-sectional diameter ranging from between about 0.030 inches or less to about 0.050 inches or more, more preferably between about 0.035 inches or less to about 0.040 inches or more, and even more preferably about 0.038 inches. The helical coil or tubular body may extend proximally from the distal tip 15 of the elongate core and may be soldered or otherwise bonded to an intermediate region 16 on the elongate core. The addition of the helical core or tubular body provides additional stiffness to the guidewire 10 thus giving the guidewire the pushability necessary to be navigated through a blood vessel or other body lumen and the trackability necessary to insert an introducer and obturator over the guidewire.

In some embodiments, the distal segment may comprise a hook-shaped region near the distal tip 15, as illustrated in FIG. 1. The hook-shaped region may be straightened in a manner known to those of skill in the art to facilitate insertion into a needle, an introducer, or other device or location.

The proximal segment 12 extends from the proximal end of the helical coil 25 to the proximal end of the elongate core 21, and is preferably comprised only of the elongate core. Thus, the proximal segment has a smaller diameter than the distal segment, and may have a constant or substantially constant diameter from the proximal end to the junction. In one embodiment, the proximal segment has the same or substantially the same mechanical properties from the proximal end of the elongate core to the junction with the helical core, e.g., the same or substantially the same rigidity, flexibility and/or stiffness along that portion of its length. The proximal segment may have a constant cross-section and a length sufficient to allow a medical device to be advanced over the guidewire through an incision in the patient's blood vessel; for example, the proximal segment may have a length ranging from about 1 cm to about 30 cm, more preferably from about 2 cm to about 20 cm, and even more preferably from about 2 cm to about 10 cm. In other embodiments the proximal segment may have lengths as described above.

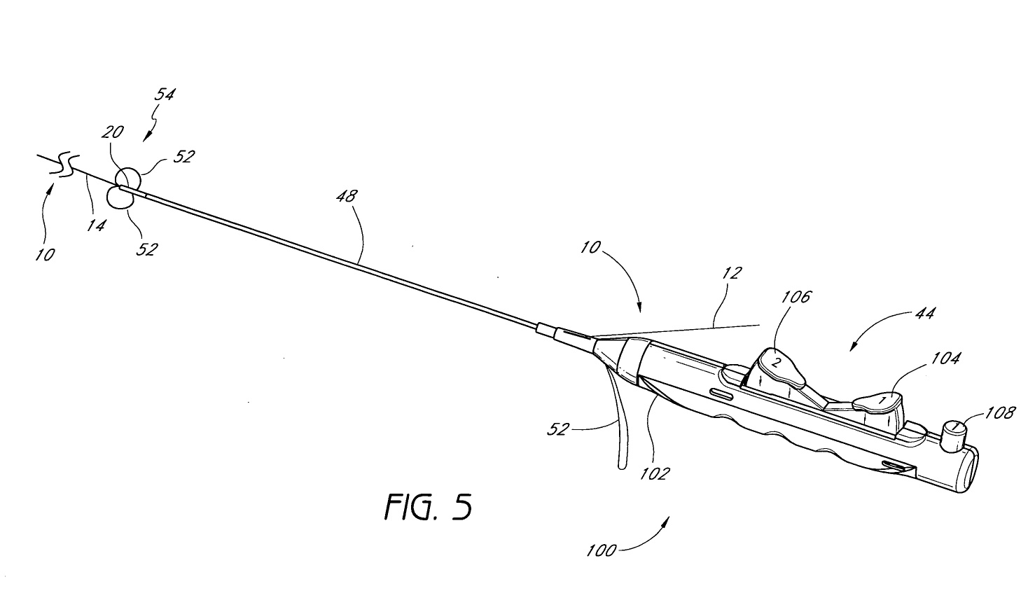

As described above, a medical device that may be advanced over a reverse tapered guidewire may be a suturing device. The medical device may be a suture delivery device, such as the suture delivery device 44 shown in FIG. 5. Further details regarding suture delivery devices and methods are described in U.S. Pat. No. 6,117,144, U.S. Pat. No. 6,562,052 and U.S. patent application Ser. No. 11/235,751, filed Sep. 27, 2005 and published on Mar. 30, 2006 as U.S. Patent Publication No. 2006/0069397, each of which is incorporated by reference herein in its entirety.

As shown in FIG. 5, the suture delivery device 44 may comprise a handle portion 100 and an elongate body 48. The handle portion 100 may comprise a housing 102, an arm trigger 104, a needle trigger 106, and an arm release button 108.

The elongate body 48 may extend from the handle portion 100 to a suture introducer head 20. A reverse tapered guidewire 10 may extend through at least a portion of the elongate body 48. As illustrated in FIG. 5, the guidewire 10 may pass through the suture delivery device 44 from a location at or near the distal end 54 to a location in or near the handle portion 100. Suture 52 may also extend through the suture delivery device 44.

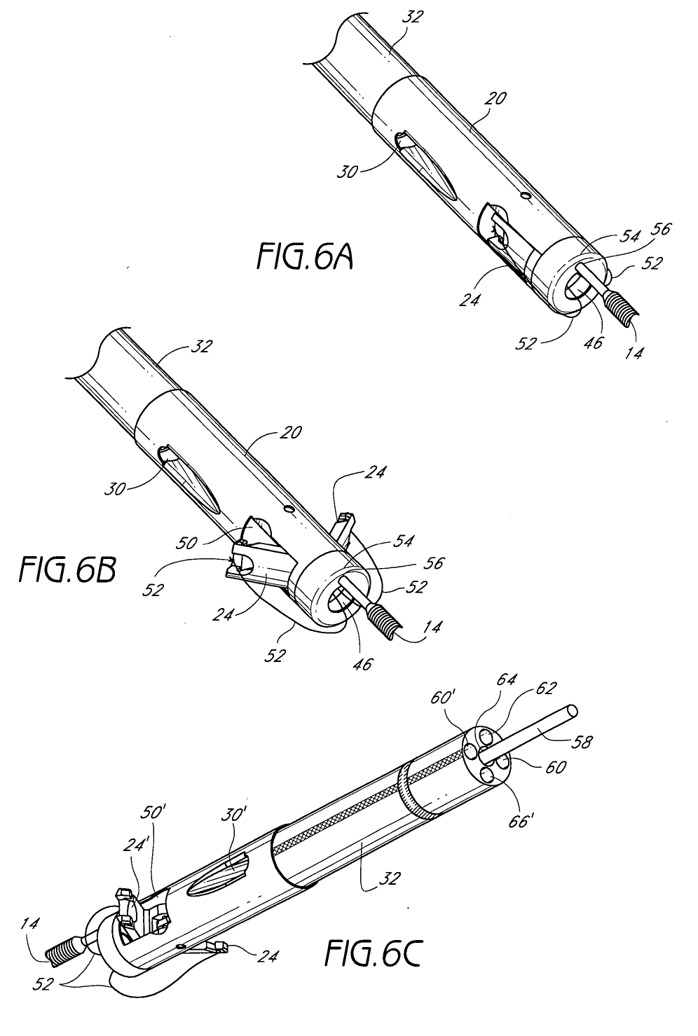

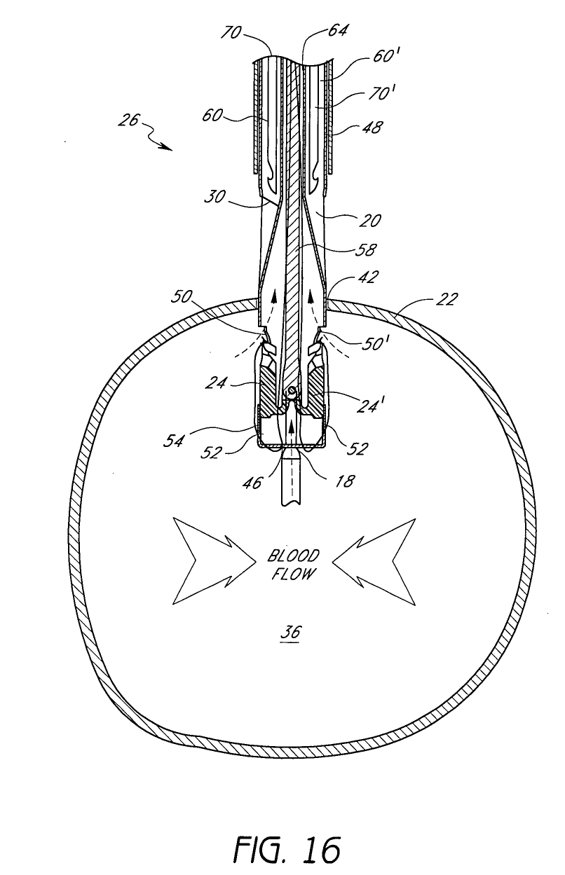

With reference to FIGS. 6A through 6C, the distal end portion of the suturing apparatus will now be described in more detail. As shown, the distal end portion may comprise the suture introducer head 20, a pair of suture arms 24, 24′, a pair of needle apertures 30, 30′, a distal end 54, a suture hole 46, a guidewire hole 56, a suture 52 and an actuating rod 58. The distal end portion may further comprise a pair of needles 70, 70′ (see FIGS. 16 through 18).

The suture arms 24, 24′ and the needles 70, 70′ may be retracted into the suture introducer head 20, as shown in FIG. 6A. This prevents the arms 24, 24′ and the needles 70, 70′ from causing tissue damage while the distal end portion passes through a biological structure.

FIGS. 6B and 6C illustrate the distal end portion of the suture delivery device 44 with the suture arms 24, 24′ partially deployed. Such deployment is achieved by partially depressing the arm trigger 104. As shown most clearly in FIG. 6B, each of the suture arms 24, 24′ may hold an end of the suture 52. The suture 52 may extend from the suture arms 24, 24′ through the suture hole 46 into the suture introducer head 20. The suture 52 may further extend through the suture introducer head 20 into the elongate body 48.

The elongate body 48 may further comprise a plurality of lumens, as illustrated in FIG. 6C. Lumens 60, 60′ may house the needles 70, 70′. Lumen 62 may house the guidewire. Lumen 64 may house the actuating rod 58. Lumen 66 may house the suture. Of course, other configurations may be used.

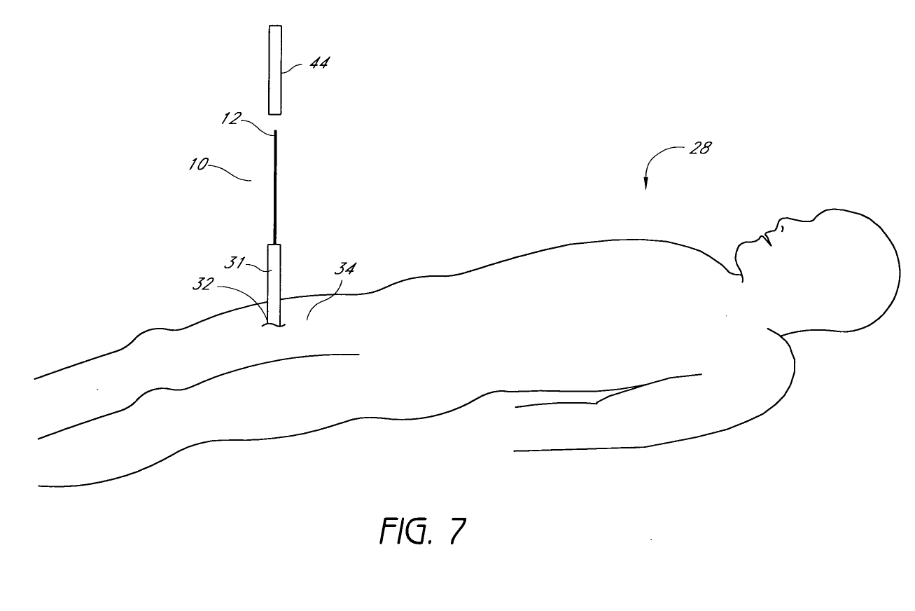

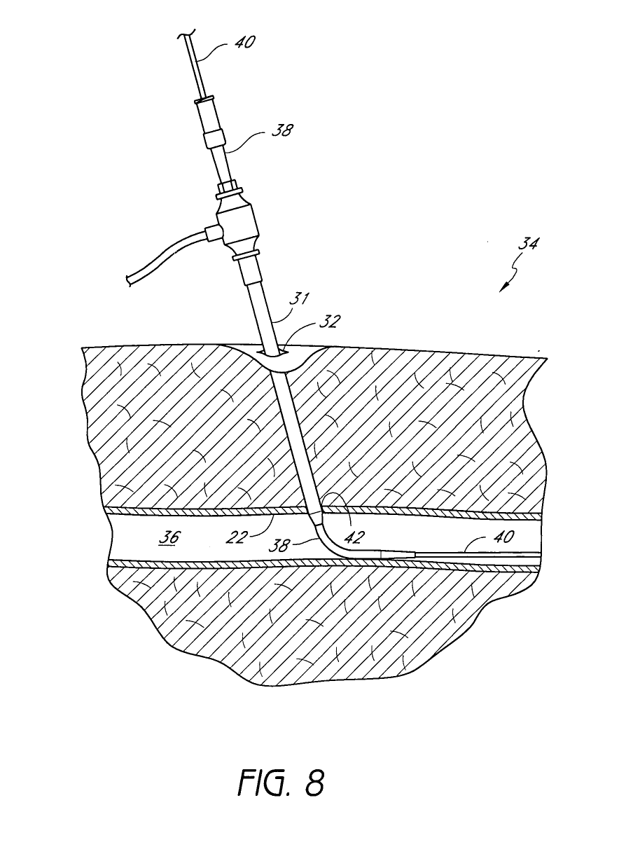

As shown in FIGS. 7-15, the tapered guidewire may be used provide sustained access to a patient's blood vessel or body cavity during and after a medical procedure, for example during suturing of a blood vessel following an interventional catheterization procedure, such as an angiogram. With reference to FIGS. 7-8, the physician makes an initial incision 32 in the upper thigh 34 of a patient 28. The physician then inserts a needle (not shown) into the incision 32. When blood bleeds back from the insertion, the physician knows the needle has pierced the femoral artery 36. The physician then inserts a guidewire 40 through the needle and through a second incision 42 into the artery 36. The guidewire 40 may be the same as reverse tapered guidewire 10, or may be a guidewire (such as a constant diameter guidewire) of a type known to those skilled in the art.

The physician may take the needle out and insert a plastic needle (not shown) over the guidewire once the guidewire is in place. The guidewire may then be taken out (not shown). With this needle in place, the physician can insert a catheter sheath introducer (CSI) 31, also called an introducer or introducer sheath. Alternatively, the guidewire 10 may remain in place during delivery and after positioning of the introducer sheath. This introducer sheath 31 is typically a single lumen catheter with a valve on its proximal end. The valve is used to prevent extraneous bleed back or to introduce medication into the patient's body.

The introducer 31 may also be inserted into the artery 36 using a tapered obturator 38, shown in FIG. 8. Once the needle has been removed, the physician may place the obturator 38 and the introducer 31 over the guidewire 40. The obturator 38 may be disposed within the introducer 31 and may be locked to the introducer 31 near a proximal end of the introducer. The obturator 38 and introducer 31 may be advanced together until the introducer 31 is at least partially within the artery 36, as shown in FIG. 8. Either or both the obturator 38 and the introducer 31 may be tapered at a distal end to facilitate entry into the artery 36.

If the guidewire 40 is sufficiently rigid, the obturator 38 will track over the guidewire 40 as it enters the artery 36, as illustrated in FIG. 8. On the contrary, the obturator may not curve over the guidewire 40 into the artery 36 and therefore damage the artery if the guidewire 40 does not provide the necessary trackability.

Once the introducer 31 is properly positioned within the artery 36, the obturator 38 and the introducer 31 may be unlocked and the obturator 38 withdrawn.

The vessel incision 42 provides access for medical instruments and probes inside the arterial vessel 36. Instruments may be inserted into artery 36 via the introducer sheath 31 to perform various procedures in the body. In some embodiments, the guidewire 40 that is used during insertion of the introducer 31 may be withdrawn after the introducer 31 has been properly positioned within the artery 36. Another guidewire (not shown), which may or may not be a reverse tapered guidewire, may then be inserted through the introducer 31 to perform a medical treatment or procedure. Alternatively, the guidewire 40 may remain in place and may be used to perform one or more medical procedures.

In one embodiment, a first guidewire may be used during insertion of the introducer 31, while a second guidewire may be used to perform a medical procedure and a third guidewire may be used to perform another or a different medical procedure. Any or all of the first guidewire, second guidewire, and third guidewire, may be a reverse tapered guidewire, as described above. A fourth, fifth, sixth guidewire may be used as desired.

Of course, any number of different guidewires may be used during a medical procedure while the introducer 31 remains properly positioned within the artery 31 to provide access to the artery 36 through the incision 42. However, access to the artery 36 through the incision 42 may be permanently lost if the introducer 31 becomes withdrawn from the artery 31 while the guidewire is also withdrawn, as will be discussed further below.

In one embodiment, the distal end 14 of a reverse tapered guidewire 10 may be advanced through the patient's thigh and femoral artery and may have a sufficient diameter to provide the rigidity necessary to advance the guidewire through the patient's blood vessel lumen to the site of the medical procedure. Optionally, the physician may take the sheath 31 out, leaving the guidewire in place providing access through the incision 42 into the blood vessel. The medical devices needed to perform a procedure may then be advanced over the guidewire to the treatment site. In one embodiment, a catheter may be advanced over the guidewire and instruments may be inserted into artery 36 via a lumen in the catheter to perform various procedures in the body

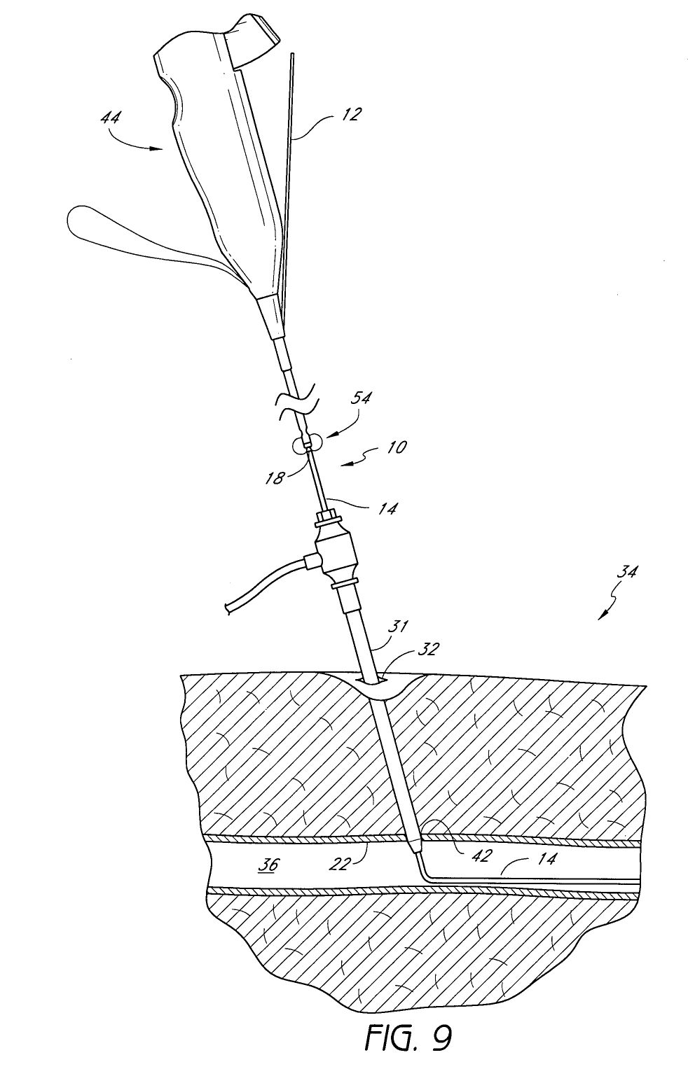

After the medical procedure, a suture delivery device 44 having a small lumen may be advanced through the CSI 31 (if still in place) and over the narrow diameter proximal segment 12 of the guidewire to the incision 42 in the artery 36. Alternatively, the guidewire used to perform the medical procedure is removed and a reverse tapered guidewire 10 is then inserted into the CSI 31. In certain embodiments, the proximal segment 12 of the guidewire may be positioned so that the intersection 18 between the proximal segment 12 and the distal segment 14 sits outside of the patient's body, while ensuring that the distal segment 14 of the guidewire remains positioned through the incision 42 providing access to the incision 42. In some embodiments, the intersection 18 between the proximal segment 12 and the distal segment 14 may be withdrawn until the intersection 18 between the proximal segment 12 and the distal segment 14 is proximally outside the introducer 31, as shown in FIG. 9. In other embodiments, the intersection 18 between the proximal segment 12 and the distal segment 14 may not need to be withdrawn from the body or introducer 31 because it has not yet been advanced into the body or introducer 31.

The suture delivery device 44 may then be loaded over the narrow proximal segment 12 of guidewire until its distal end 54 abuts against the transition 18 to the larger diameter distal segment 14, as illustrated in FIG. 9. In some embodiments, the suture delivery device 44 may be loaded over the proximal segment 12 of guidewire 10 before the guidewire is inserted.

Preferably, the guidewire lumen 62 (FIG. 6C) and/or the guidewire hole 56 (FIGS. 6A and 6B) of the suture delivery device 44 is smaller than the diameter of the distal segment 14, such that suture delivery device cannot be advanced beyond the transition 18. The distal segment 14 of the guidewire 10 is preferably configured to have a length that remains extended through the incision 42 in the artery 36 when the proximal segment 12 is withdrawn from the body. In some embodiments, the distal segment 14 of the guidewire 10 may have a length that remains extended through the incision 42 in the artery 36 when the proximal segment 12 is withdrawn from the introducer 31.

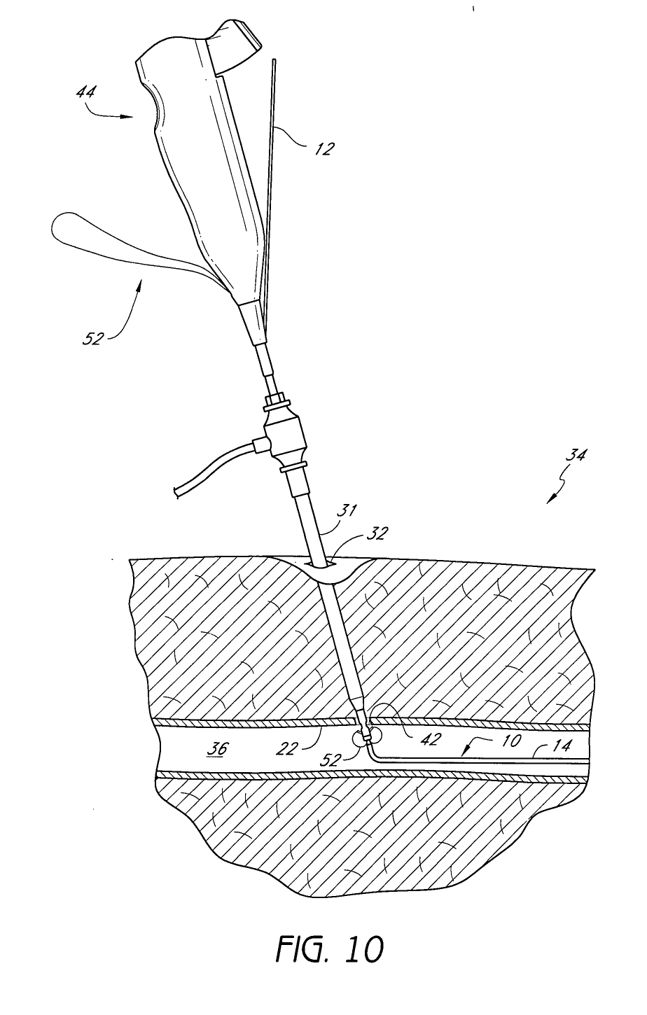

Thus, when the suture delivery device 44 is advanced distally through the patient's thigh, it moves with the guidewire and pushes through tissue to get to the incision 42. The larger diameter distal segment 14 of the guidewire 10 preferably has sufficient rigidity to guide the device 44 to the incision site 42. The CSI 31 (if still in place) may be withdrawn from the vessel, as illustrated in FIG. 10, in order to permit activation of the suture delivery device 44 to deliver suture 52 to the vessel wall 22 surrounding the incision 42 in the artery 36, as will be described further below (see FIGS. 16-18).

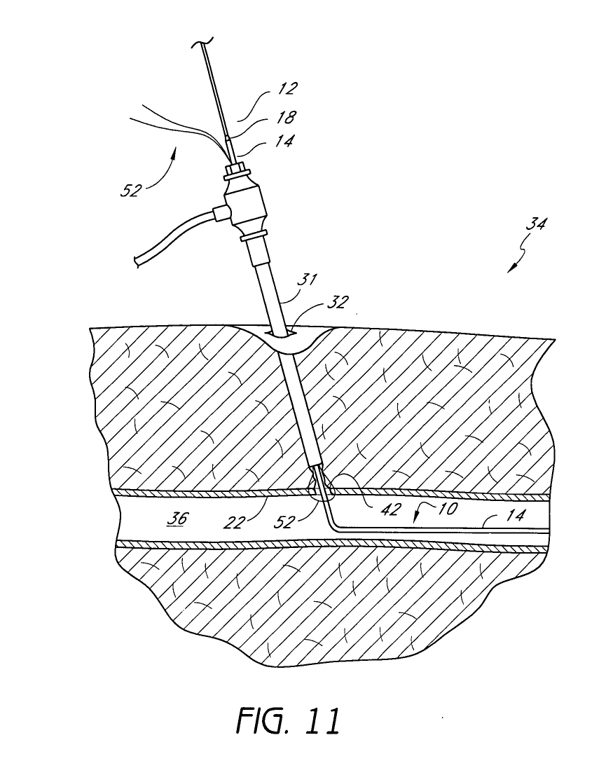

The physician may then withdraw the device 44 out of the blood vessel 36 and out of the patient's thigh 34, as illustrated in FIG. 11. The suture delivery device 44 and guidewire 12 may be withdrawn together or the device 44 may be withdrawn without withdrawing the guidewire 10. In some embodiments, the device 44 may be withdrawn off the wire.

After the device 44 is withdrawn (with the guidewire 10 still in place in the incision 42 and the CSI 31 still possibly in the tissue of the patient), the physician pulls the ends of the suture and closes the main vessel incision 42 by pulling the sutures tight and tying or applying a knot. The physician may tie a fisherman's knot or an improved clinch knot with the ends of the suture and slide or push the knot down through the CSI 31 to the vessel incision 32. The physician may tie and push the knot(s) by using any suitable suture knot tying and/or cinching apparatus including an apparatus disclosed in Applicant's application entitled METHOD AND APPARATUS FOR TYING SUTURE KNOTS, Ser. No. 09/923,108, filed Aug. 6, 2001, the entirety of which is hereby incorporated by reference. Alternatively, the physician may tie at least one knot by hand and then cinch the knot by using a knot cinching device, such as an apparatus taught by Applicant's application titled KNOT PUSHER, Ser. No. 09/571,759, filed May 15, 2000, which is incorporated herein by reference in its entirety. Still, the physician may choose to fasten a small, circular or flat stainless steel clip (not shown) to the ends of the suture and slide the clip down through the CSI 31 to the vessel incision 42 to close the incision 42. Other embodiments for tying and placing knots are described in Applicant's application entitled METHOD AND APPARATUS FOR HOLDING SUTURE ENDS TO FACILITATE TYING OF KNOTS, Ser. No. 60/683,701, filed May 23, 2005, and Applicant's application entitled METHOD AND APPARATUS FOR HOLDING SUTURE ENDS TO FACILITATE TYING OF KNOTS, Ser. No. 11/438,619, filed May 22, 2006, published Dec. 14, 2006 as U.S. Patent Publication No. 2006/0282102, the entirety of each of which is hereby incorporated by reference. The physician may apply a knot to secure the suture ends as described in U.S. patent application Ser. No. 11/455,894, filed Jun. 19, 2006, entitled METHOD AND APPARATUS FOR APPLYING A KNOT TO A SUTURE, published Jan. 11, 2007 as U.S. Patent Publication No. 2007/0010829, the entirety of which is hereby incorporated by reference.

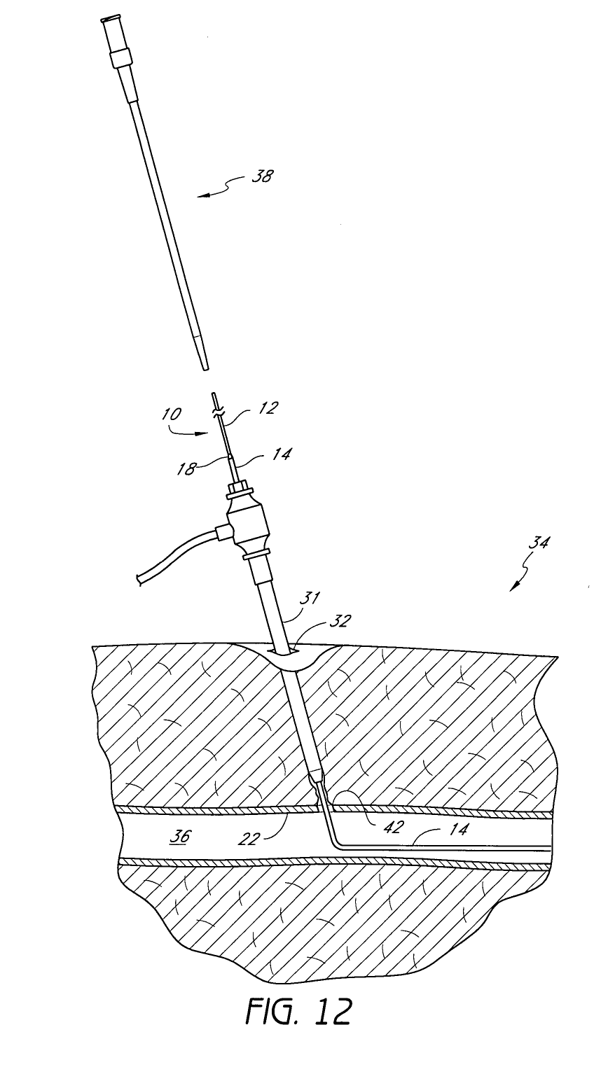

Typically, the suture will adequately close the incision. However, in certain high risk patients, the suture may fail. For example, in patients with extensive plaque build up along the arterial wall, the sharp edges of the plaque may cut the suture as it is being pulled tight about the opening. Alternatively, in certain patients with thin arterial walls, the force of the suture against the arterial wall as it is being pulled tight may cause the suture to be pulled through the wall and thus prevent closure of the wall opening. With the guidewire 10 still in place in the incision 42, but the CSI withdrawn proximally from the incision, as shown in FIG. 12, the incision can be re-accessed to deliver another suture. Thus, if the first suture should fail for one of the above mentioned reasons, or if it is simply desired to place additional sutures, the physician may re-insert the suture delivery device over the guidewire 10 and re-access the incision 42 to deliver a second suture to properly close the incision.

In one embodiment, the guidewire 10 may be withdrawn from the patient after the suture 52 has been pulled tight and the suture 52 has not been cut by plaque, torn out, or otherwise failed. Alternatively, the physician may leave the guidewire 10 in the incision 42 in the artery 36 while pushing the knot toward the incision or otherwise placing the knot. The physician may withdraw the guidewire 10 before cinching the knot. The physician may then cut the unused ends (extra length) of the suture 52 and remove the cut portions. The physician may then remove the CSI 31 from the patient's thigh.

If the suture fails, the guidewire 10 may allow the physician to re-access the incision 42. In some embodiments, an obturator may be used over the guidewire 10 to re-access the incision 42 with the introducer 31. The physician may position the guidewire 10 such that the intersection 18 between the proximal segment 12 and the distal segment 14 is outside the patient and the introducer 31, as shown in FIG. 12. In some embodiments, the intersection 18 between the proximal segment 12 and the distal segment 14 need not be outside the patient to advance the obturator 38 into the artery 36.

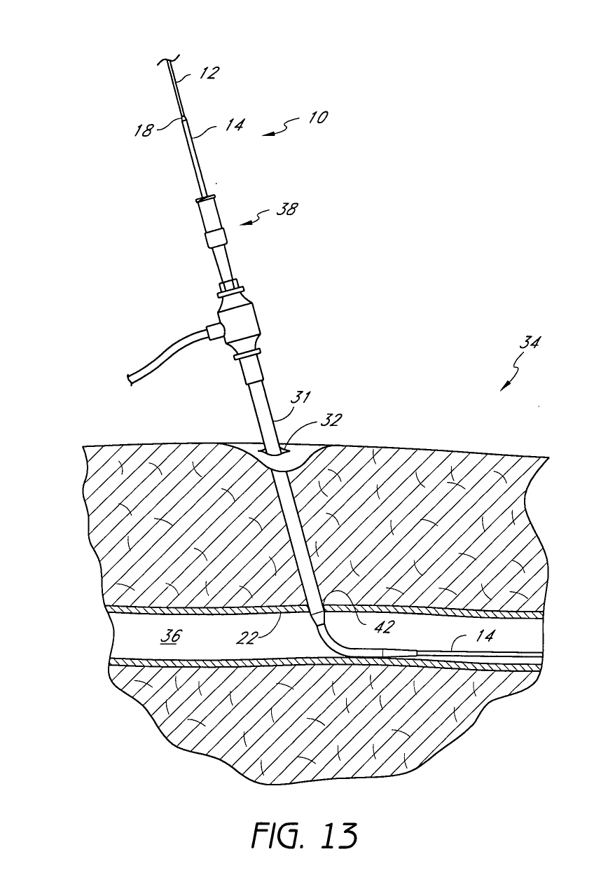

The obturator 38 may be advanced over the guidewire 10 through the introducer 31. The obturator 38 and introducer 31 may lock together as described above as the obturator is advanced through the introducer 31. The obturator 38 and the introducer 31 may then be advanced, either together or independently, through the incision 42 into the artery, as illustrated in FIG. 13. Ether the obturator 38, the introducer 31, or both may have a tapered distal end to facilitate entry into the artery 36.

The intersection 18 between the proximal segment 12 and the distal segment 14 of the guidewire 10 may be located proximally from the distal end of the obturator 38 to cause the obturator to bend as it enters the artery 36 so as to avoid causing trauma to the artery 36. The obturator 38 may then be withdrawn to permit access to the artery 36 through the introducer 31. Alternatively, the artery 36 may be accessed over the guidewire 10 without reinsertion of the introducer 31 into the artery 36 using the obturator 38. The guidewire 10 may provide sufficient trackability or pushability to allow a medical device to access the artery 36 through incision 42.

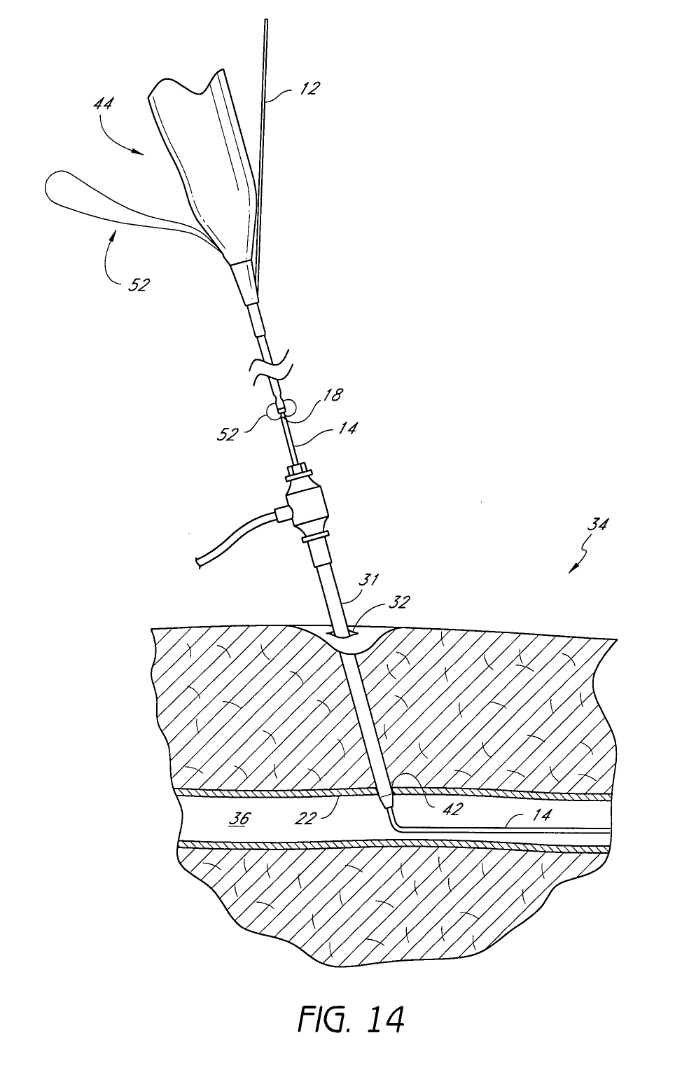

As shown in FIG. 14, the same or a different suture delivery device 44 may be advanced over the guidewire 10. The intersection 18 between the proximal segment 12 and the distal segment 14 may be positioned outside the patient and the introducer 31, as shown in FIG. 14. The device 44 may be advanced until the distal end 54 of the device 44 abuts the transition 18 between the proximal segment 12 and the distal segment 14.

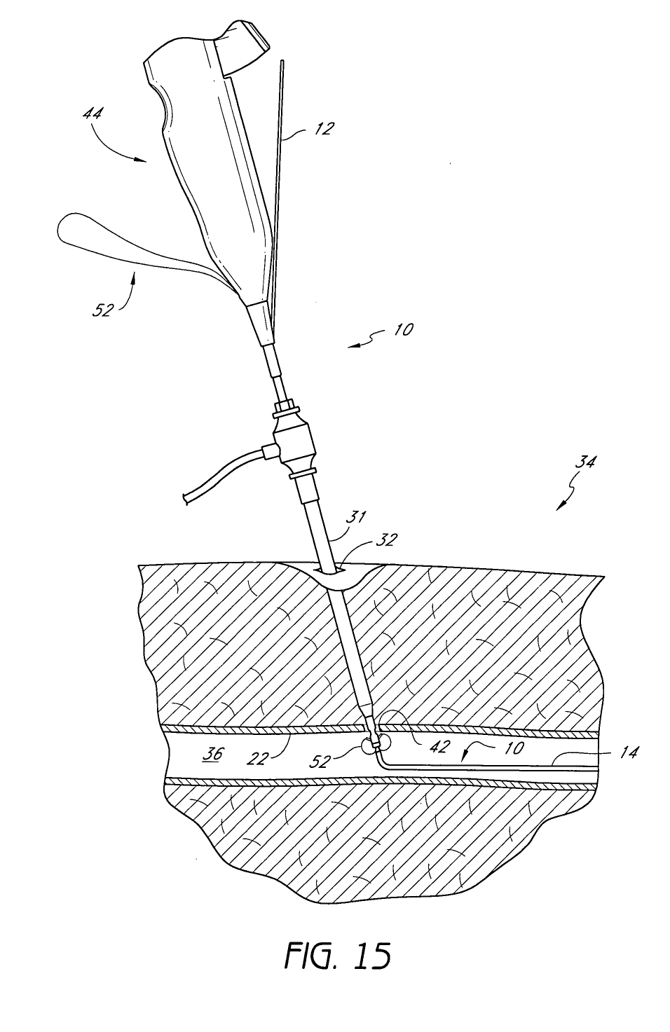

Once the distal end 54 of the suture delivery device 44 is positioned within the artery 36, the introducer 31 may be withdrawn, as illustrated in FIG. 15, to permit placement of a suture 52 in the tissue surrounding the incision 42.

While the suture introducer head 20 is inserted into the artery 36, as shown in FIG. 16, the actuating rod 58 holds the suture arms 24, 24′ in a recessed state within the suture introducer head 20. The suture arms 24, 24′, hold a looped end of the suture 52, as illustrated in FIGS. 6A through 6C.

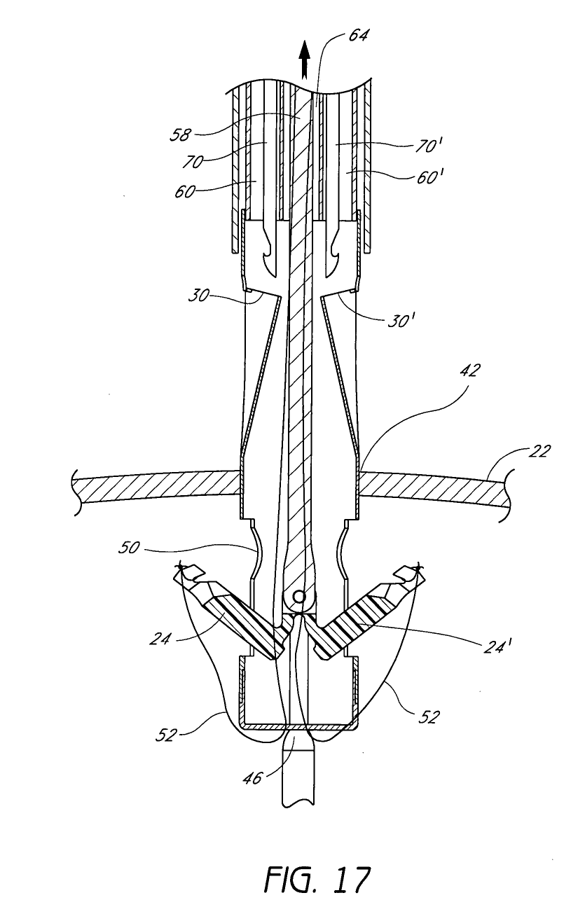

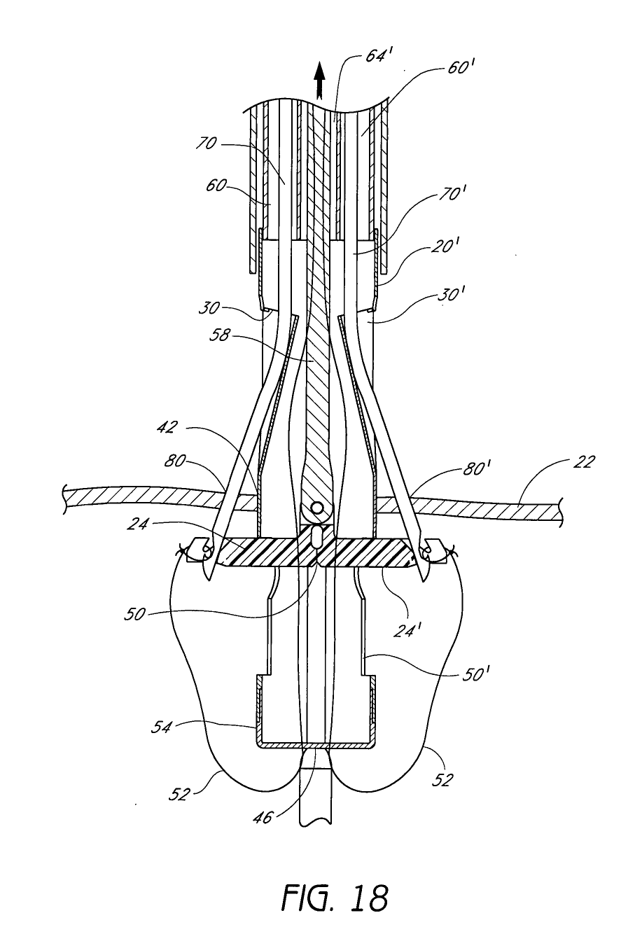

Once the distal portion 26 of the device 44 is properly positioned within the artery 36, the physician depresses the arm trigger 104 (FIG. 5) to deploy the suture arms 24, 24′ as shown in FIG. 17. As the physician continues depressing the arm trigger 104, the suture arms 24, 24′ are moved into a fully deployed state as illustrated in FIG. 18. With the suture arms 24, 24′ in this fully extended position, the physician may gently slide the suture delivery device 44 proximally so that the suture arms 24, 24′ contact the interior surface of the vessel wall 22.

At this juncture, the physician depresses the needle trigger 106 (FIG. 5) on the handle portion 100 to distally advance the needles 70, 70′ and capture the ends of the suture 52 from the suture arms 24, 24′. During advancement, the needles 70, 70′ penetrate the vessel wall 22 at an angle, thereby creating the needle incisions 80, 80′ on opposite sides of the incision 42.

Pressure on the needle trigger 106 is released to retract the needles 70, 70′ proximally. This motion causes the needles 70, 70′ to withdraw into the needle lumens 60, 60′ with the looped ends of the suture 52. As the needles 70, 70′ pull proximally on the looped ends of the suture 52, tension in the suture 52 causes additional segments of the suture 52 to feed through the hole 46 at the distal end 54 of the suture introducer head 20, into the artery 36 and through the needle incisions 80, 80′.

Once the needles 70, 70′ have been retracted into the needle lumens 60, 60′, the physician depresses the arm release button 108 (FIG. 5) to release the arm trigger 104. Once the arm trigger 104 is released, the suture arms 24, 24′ retract into the recessed state within the suture introducer head 20, as shown in FIG. 16. In the recessed state, the suture arms 24, 24′ are substantially parallel with the hollow elongate body 32, and the exterior surfaces of the suture arms 24, 24′ are substantially flush with the exterior surface of the introducer head 20. This reduces the likelihood that the suture arms 24, 24′ will snag or catch on the vessel wall 22 or the flesh during withdrawal. With the suture arms 24, 24′ and the needles 70, 70′ returned to the recessed state, the device 44 is ready for removal from the artery 36.

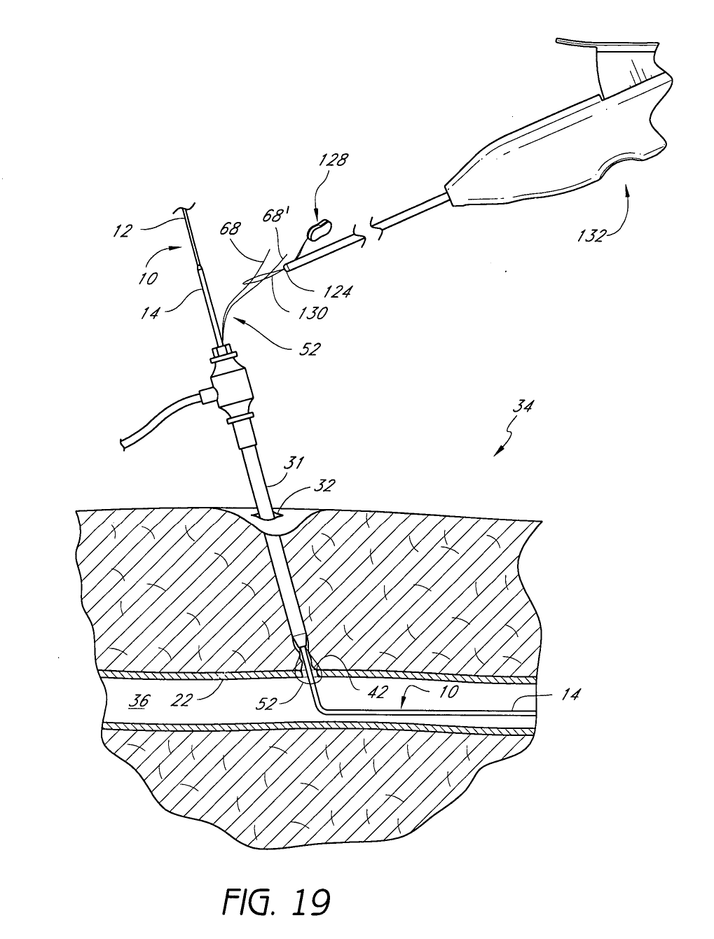

After the suture delivery device 44 is withdrawn from the patient, the suture 52 may extend through the vessel wall 22 surrounding the incision 42 while a pair of suture ends 68, 68′ of the suture 52 extend out of the body through the introducer 31, as illustrated in FIG. 19.

The physician may close the incision 42 using any of the methods or apparatuses described above. In one embodiment, the sutures ends 68, 68′ may be passed through the loop 130 of a threader 128, as shown in FIG. 19. The threader is preloaded into a knot placement device 132. The threader 128 may be pulled proximally to dispose suture 52 in the knot placement device 132. Further details regarding knot placement devices and methods are described in U.S. patent application Ser. No. 11/455,894, filed Jun. 19, 2006, entitled METHOD AND APPARATUS FOR APPLYING A KNOT TO A SUTURE, published Jan. 11, 2007 as U.S. Patent Publication No. 2007/0010829, the entirety of which is hereby incorporated by reference.

The physician may pull the suture 52 tight to close the incision 42. The guidewire 10 may be withdrawn if the suture 52 has been pulled tight and the suture 52 has not been cut by plaque, torn out, or otherwise failed. The suture 52 may be held in tension, by hand or otherwise, while the knot placement device 132 is advanced through the introducer 31. In other embodiments, the knot placement device 132 may be advanced through the introducer 31 beside the guidewire 10 or over the guidewire 10.

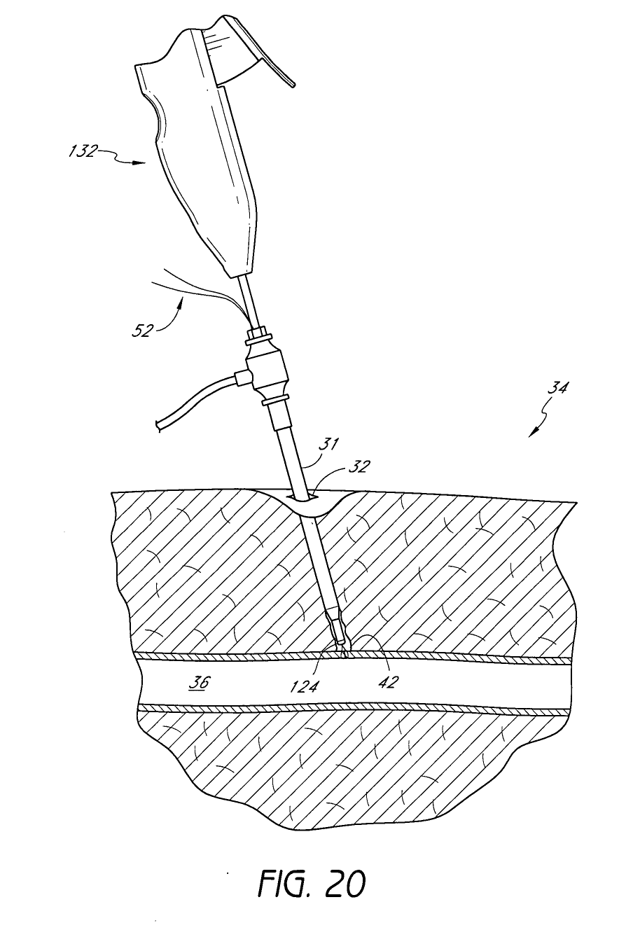

The knot placement device 132 may be advanced until a knot body 124 contacts or is near to the vessel wall 22, as illustrated in FIG. 20. The knot placement device 132 may be actuated to secure the suture 52 within the knot body 124, eject the knot body 124 from the knot placement device 132, and sever an excess portion of suture 52. The knot placement device may then be removed, leaving the knot in place against the tissue portions. The introducer 31 may then be removed from the patient and incision 32 closed.

In an alternative embodiment, the method of advancing a suture delivery device 44 over the narrow proximal segment 12 of a guidewire positioned in the patient's blood vessel may be used to pre-load one or more sutures at the incision site. Initially, the distal section 14 of the guidewire 10 may be advanced through an incision 42 into the patient's blood vessel 36. After performing any desired treatment over the guidewire, the proximal segment 12 of the guidewire may then be withdrawn from the patient's body such that the suture delivery device 44, or other medical device having a small lumen, may be loaded over the narrow proximal segment 12 of the guidewire 10 while the distal segment 14 of the guidewire 10 remains in position through the incision 42 providing access to the incision 42. Once the suture delivery device 44 has been loaded over the narrow proximal segment 12 of the guidewire, the guidewire 10 may be advanced further through the blood vessel to advance the suture delivery device 44 to the incision 42.

The suture delivery device 44 may then be used deliver the suture 52 and be withdrawn from the blood vessel and the patient's body over the guidewire 10. The sutures 52 may then be laid aside without being pulled closed and tied. The suture delivery device 44 may be reloaded with another suture and re-introduced to the incision 42 over the proximal segment 12 of the guidewire to deliver another suture to the exact same incision. Alternatively, a different suture delivery device may be used rather than reloading the same suture delivery device. The suturing device 44 may then again be withdrawn from the blood vessel 36 and the patient's body over the guidewire 10 and the suture 52 laid aside without being pulled closed and tied. This procedure may be repeated as many times as necessary to provide enough sutures to adequately close a larger incision site.

Alternatively, once one or more sutures have been pre-loaded at the incision 42, the guidewire 10 may be used by the physician to re-access the incision 42 to deliver additional medical instruments to the blood vessel 36 and perform a medical procedure within the blood vessel 36. In certain embodiments, the doctor may introduce a larger catheter over the guidewire 10 to enlarge the incision in the blood vessel 36 in order to perform the medical procedure. Once the medical procedure is complete, the pre-loaded sutures may be pulled tight and knotted to close the enlarged incision.

One of skill in the art will appreciate that some medical devices will not accommodate passage therethrough of a guidewire that is sufficiently rigid to provide re-access to a location. It may nevertheless be desirable to maintain the ability to re-access a location even when using such a medical device. In some embodiments, a reverse tapered guidewire may permit such a medical device to be advanced over a relatively small proximal segment of the guidewire, while a relatively large distal segment is within the body. In this manner the ability to re-access a location may be preserved even when using a medical device that will not accommodate passage therethrough of a guidewire large enough to permit re-access.

A reverse tapered guidewire and the methods described herein may also be used in some embodiments to access or re-access locations within the body other than a blood vessel. For example, a reverse tapered guidewire may facilitate access or re-access through an abdominal wall, dural sac, vaginal tube, intestinal tube, spinal canal, or any other location where a wire may pass through biological tissue to allow re-access to the location. Embodiments of the present invention could be similarly used to access or re-access a patent ductus arteriosus, a patent foramen ovale, a heart defect, a puncture wound in the skin, and other bodily tissues.

While a number of preferred embodiments of the invention and variations thereof have been described in detail, other modifications and methods of using and medical applications for the same will be apparent to those of skill in the art. Accordingly, it should be understood that various applications, modifications, combinations, sub-combinations and substitutions may be made of equivalents without departing from the spirit of the invention or the scope of the claims.

What is claimed is:

1. A method of delivering an intravascular medical device to a body lumen of a patient, comprising:
advancing a guidewire through an access site on the patient into the lumen of a blood vessel, the guidewire having a proximal region and a distal region, wherein the proximal region has a cross-sectional diameter smaller than a cross sectional diameter of the distal region;
advancing an intravascular device over at least the proximal region of the guidewire into the lumen of the blood vessel;
performing an operation at a treatment site within the patient with the intravascular device;
withdrawing the intravascular device from the patient; and
introducing the same or a different intravascular device over the proximal segment of the guidewire.

2. The method of claim 1, wherein the distal segment of the guidewire has a cross-sectional diameter ranging from 0.030-0.050 inches and wherein the proximal segment of the guidewire has a diameter ranging from 0.010-0.018 inches.

3. The method of claim 2, wherein the intravascular medical device has a lumen having a cross-sectional diameter smaller than the cross-sectional diameter of the distal region of the guidewire.

4. The method of claim 1, wherein the treatment site is an incision site, and wherein performing an operation comprises suturing the incision site with a suturing device.

5. The method of claim 4, wherein the same or a different intravascular device is a suturing device.

6. The method of claim 1, wherein the intravascular device is advanced over the guidewire with its distal end adjacent to an intersection between the proximal region and the distal region of the guidewire and then advancing the intravascular device with the proximal region into the lumen of the blood vessel.

7. The method of claim 1, further comprising advancing the same or a different intravascular device into the lumen of the blood vessel.

8. A method of providing access to a blood vessel of a patient, comprising:
advancing a guidewire through an access site in the patient's tissue and through an incision in a lumen of a blood vessel, the guidewire having a proximal region and a distal region, wherein the proximal region has a cross-sectional diameter smaller than a cross sectional diameter of the distal region;
holding a proximal portion of the wire outside of the patient to maintain the position of the distal region of the guidewire within the incision in the blood vessel;
advancing a medical device over the proximal region of the guidewire until it abuts the beginning of the larger diameter distal region; and
moving the guidewire and the medical device distally through the patient's tissue past the incision in the body lumen.

9. The method of claim 8, further comprising performing an operation with the medical device at the incision.

10. The method of claim 8, further comprising withdrawing the medical device from the patient and removing the medical device from the proximal end of the guidewire while maintaining the distal end of the guidewire in position in the incision in the body lumen.

11. The method of claim 8, wherein the medical device is a suturing device.

12. A method of performing a medical procedure, comprising:
delivering a guidewire into a patient, the guidewire having a proximal segment and a distal segment, wherein the proximal segment has a smaller diameter than that of the distal segment, such that the proximal segment extends outside the patient after the guidewire is delivered; and
advancing a medical device over the guidewire into the patient.

13. The method of claim 12, wherein the medical device is advanced until a distal end of the medical device engages a transition between the proximal and distal segments.

14. The method of claim 12, wherein the medical device has a lumen that is smaller than the diameter of the distal segment.

15. A method of placing at least one suture, comprising:
inserting a guidewire into a body opening, the guidewire comprising a proximal segment and a distal segment, the proximal segment having a smaller diameter than the distal segment;
advancing a suture delivery device at least partially over the proximal segment of the guidewire to the opening;
applying at least one suture to the opening using the suture delivery device; and
withdrawing the suture delivery device from the patient's body over the guidewire while retaining the guidewire within the body opening.

16. The method of claim 15, further comprising performing a medical treatment other than delivering a suture before advancing the suture delivery device over the guidewire.

17. The method of claim 15, further comprising advancing the suture delivery device over the proximal segment of the guidewire to the opening again after retracting the suture delivery device and applying another suture to the opening.

18. The method of claim 15, further comprising advancing a different suture delivery device over the proximal segment of the guidewire to the opening after retracting the suture delivery device and applying another suture to the opening.

19. The method of claim 15, further comprising applying a knot to the at least one suture applied to the opening.

20. The method of claim 15, wherein the suture delivery device applies at least one suture by piercing tissue with at least one needle.

21. The method of claim 15, wherein the suture delivery device is advanced until its distal end abuts an intersection between the proximal and distal segments and then advancing the suture delivery device with the guidewire into the opening.

22. The method of claim 15, wherein the body opening is a blood vessel.

23. The method of claim 22, further comprising advancing an introducer over the guidewire into the blood vessel.

24. The method of claim 23, wherein the introducer is advanced over the guidewire into the blood vessel after withdrawing the suture delivery device from the patient's body over the guidewire while retaining the guidewire within the body opening

25. A guidewire, comprising:
a proximal segment and a distal segment, wherein the proximal segment has a smaller diameter than that of the distal segment;
wherein the proximal segment has a substantially constant diameter and substantially consistent mechanical properties from a proximal end of the guidewire to a transition between the proximal and distal segments.

26. The guidewire of claim 25, wherein the proximal segment has a diameter of between about 0.010 and 0.018 inches and the distal segment has a diameter of between about 0.030 and 0.050 inches.

27. The guidewire of claim 25, in combination with a suture delivery device having a lumen configured to permit the proximal portion to pass therethrough, but inhibiting the distal portion from entering the lumen.

28. The combination of claim 27, wherein the suture delivery device is adapted to deliver a suture using at least one needle.
